(19) United States
(12) Patent Application Publication
(10) Pub. No.: US 2023/0293204 A1
(43) Pub. Date: Sep. 21, 2023

(54) CERVICAL DILATOR

(71) Applicant: Celestial Life Sciences, LLC, Washington, DC (US)

(72) Inventor: Matthew Reeves, Washington, DC (US)

(21) Appl. No.: 17/820,671

(22) Filed: Aug. 18, 2022

Related U.S. Application Data
(60) Provisional application No. 63/234,493, filed on Aug. 18, 2021.

(51) Int. Cl. A61B 17/42

(52) U.S. Cl. CPC A61B 17/42; A61B 2017/00557

(57) ABSTRACT

A cervical dilator is positioned in the cervix of a subject, as an outpatient procedure. Then, the cervical dilator is inflated by adding water to begin expansion of an expandable material located within the patient’s cervix. The expandable material expands the cervical dilator balloon at a rate of 1-3 mm/hr until a cervical dilation to 3 cm is obtained, typically at the subject’s home overnight prior to starting induction of labor or other procedures where cervical dilation in needed.

### CROSS-REFERENCE TO RELATED APPLICATIONS

This application claims the benefit of U.S. Provisional Pat. Application No. 63/234,493 filed Aug. 18, 2021, and the complete contents thereof are herein incorporated by reference.

### BACKGROUND

A cervical dilator is needed for many obstetrics and gynecology (ob/gyn) procedures. For example, in the United States there are currently approximately six million deliveries per year. Of those, there are an estimated 1.6 million inductions of labor per year. Induction is currently expensive and slow. To address this concern, there are currently the Foley Balloon and the Cook Balloon that are being used to address induction in labor. Both the Foley and Cook Balloons are used almost exclusively in-patient. An out-patient cervical prep is now being marketed by Medicem using multiple Dilapan-S. This prep is difficult to place and may have high risk of complications. As such, few obstetricians use it in the US and virtually none use it outside the US.

The Cook Balloon is a minor improvement over the Foley Balloon. The Cook Balloon is a silicone double balloon, whereas the Foley Balloon has a single balloon. The Cook Balloon is water-filled; however, with the Cook Balloon water can only dilate based on the elasticity of the balloon wall material. The balloon material elasticity is fixed with no control of shape, it has limited control of dilation, and pressure starts high then decreases.

### SUMMARY

There is a need for a reliable, safe way to achieve good cervical dilation at time of admission for induction. With the current products, one must dilate the cervix very slowly with constant or decreasing force after hospital admission. If the force is too high initially from the device, it will be painful and uncomfortable for the patient. Saving time, particularly for in-patient care, would provide significant cost advantages for the care provider, and likely would be preferred by patients.

According to an aspect of some embodiments of the invention, the new cervical dilator can be used overnight as an out-patient procedure. Overnight the cervical dilator balloon exerts pressure on the cervix to slowly open it. This pressure produces a change of roughly 1 mm/hr initially and is slightly greater than 1-2 mm/hr at the end of 24 hrs. Thus, when the patient comes in for in-patient induction, the cervix of the patient is reliably 3 cm or more at the start of induction. This provides savings of hours of in-patient time.

According to another aspect of some embodiments of the invention, the new cervical dilator has one balloon chamber having multiple segments. An internal segment is above the cervix (in the lower uterus) and keeps the cervical dilator balloon in place and serves as a small portion of the water reservoir. The middle segment is positioned in the cervix and holds an expandable hydrogel which applies force to the cervix as the hydrogel expands. A vaginal segment (outside the cervix, in the vagina) is elastic and is used as a main reservoir of water for the cervical segment.

A significant feature of an exemplary dilator is that water is used as the fluid that expands the hydrogel in the cervical segment instead of bodily fluids. The dual purpose of the balloon which surrounds the hydrogel is (1) to ensure proper positions of the device within the cervix and (2) hold water for the hydrogel to absorb. Water is significant since hydrogels absorb roughly six times more water than bodily fluids containing salts (primarily sodium and chloride). Thus, the same amount of hydrogel can yield a much larger effect on cervical dilation when surrounded by water instead of absorbing the naturally occurring bodily fluids, the availability of which can vary among patients.

In some embodiments, the cervical dilator balloon may be 8-10 cm in length, where the cervical segment plus portions of the vaginal segment and uterine segment immediately adjacent the cervical segment is 5-6 cm in length, and each of the external segment and the internal portion may be 1-3 cm in length where the lengths of the external segment and internal portion may be the same or different from each other. In an uninflated state the cervical balloon dilator may be, for example, less than one centimeter in diameter (e.g., 7-9 mm). In some embodiments, the cervical dilator balloon has a single valve, a uterine segment that is 1-3 cm long, a cervical segment that is 3-5 cm long, and a vaginal segment which is 2-4 cm long, where the balloon formed by the segments is all one chamber.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a generalized configuration of an exemplary cervical dilator.

FIG. 2 is a three part drawing of the cross-sectional view (top), inflated configuration (middle), and deflated configuration (bottom) of another exemplary cervical dilator.

FIG. 3 is a schematic of a combination inflation value/removal loop.

FIG. 4 is an exemplary medical device for cervical dilation.

FIG. 5 is a cross-sectional view taken from FIG. 4.

FIG. 6 is an exploded view of the medical device of FIG. 4.

FIGS. 7A, 7B, 7C, and 7D illustrate schematically an exemplary method of dilating a cervix.

### DETAILED DESCRIPTION

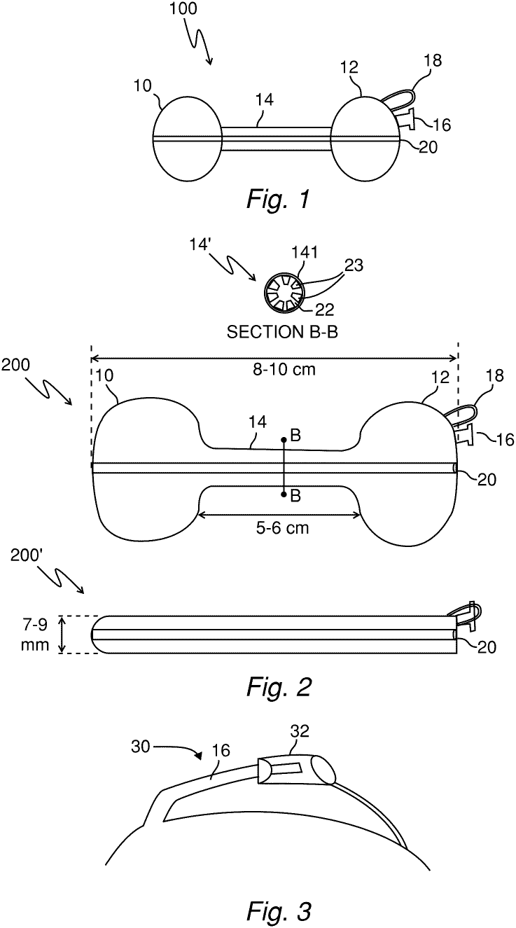

FIG. 1 shows a generalized configuration of an exemplary medical device 100 for cervical dilation. The medical device 100 may also be referred to interchangeably in this disclosure as a cervical dilator balloon or a cervical dilator, or simply a dilator. The cervical dilator 100 may have one or more individually distinguishable segments along its length. These segments may be referred to as a uterine segment 10, a vaginal segment 12, and a cervical segment 14. A uterine segment 10 may sometimes referred to in this disclosure as an inner segment (in reference to its placement inside the uterus) or distal segment (in reference to it being the segment most distal from the clinician during placement). A vaginal segment 12 may sometimes be referred to in this disclosure as an external segment (in reference to its placement outside the uterus) or proximal segment (in reference to it being the segment most proximal or nearest the clinician during placement). A cervical segment 14 may sometimes be referred to in this disclosure as a middle segment (in reference to its position between the segments at either end of the dilator 100). Furthermore, “segment” and “balloon” may be referred to interchangeably for some embodiments. “Balloon” may generally be used to refer to a structure or part of a structure which is configured so as to be capable of changing in size and/or volume, e.g., swell, grow, shrink, contract).

FIG. 1 shows a uterine segment 10 and an vaginal segment 12 are positioned at opposite ends of the cervical segment 14. The uterine segment 10 is configured to hold the cervical dilator 100 in proper position during use. An exemplary uterine segment 10 may be about 1 cm larger in radius than the cervical segment 14 and may be made from a non-elastic or minimally elastic material. The cervical segment 14 functions to exert pressure radially outward to dilate the cervix. Notably the cervical os resists dilation the most, and the cervical segment 14 may be structurally reinforced in order to maintain pressure on the internal os. The vaginal segment 12 may serve as a fluid reservoir for the cervical segment 14 as it expands. That is, during an exemplary cervical dilation procedure, the vaginal segment 12 holds much (e.g., most, more than 50%) of the water supplied to the interior of the dilator 100 at the outset of the procedure, but such water over the course of the dilator’s use (e.g., up to 36 hours) is mostly absorbed by the cervical segment, in particular an expandable material in the cervical segment. The vaginal segment 12 may be configured to act as a fluid reservoir by maintaining limited pressure (e.g., according to the material properties of the walls of the vaginal segment) that causes or at least contributes to expansion of the middle segment 14. On the vaginal segment 12, the dilator 100 may comprise an inflation valve 16 through which fluid is added to the vaginal segment 12 and begin to inflate the segments 14 and 10 as well so that the vaginal segment 12 is able to exert pressure on the cervical segment 14. The inflation valve 16 may be kept close to the vaginal segment 12 so that the valve 16 does not protrude from the vagina of the patient.

The dilator 100 may further comprise a removal loop 18 which may be connected to the external balloon 12 to facilitate removal of the cervical dilator balloon from the cervix of the patient. In some embodiments, the removal loop 18 may be attached to the inflation valve 16 so that the inflation valve 16 may be brought out of the patient first for deflation if needed, and to hold the inflation valve 16 close to the external balloon 12 when the external balloon 12 is inflated. A channel 20 may extend through the cervical dilator 100 to allow a stylet to be placed therein and assist with insertion of the cervical dilator 100 into the cervix.

FIG. 2 shows a partially inflated generalized configuration of a cervical dilator 200 (segment 14 is yet to substantially enlarge), above a fully deflated generalized configuration of the cervical dilator 200′, and below a cross-sectional view 14′ of the middle segment 14. The dilator 200 differs from the dilator 100 in certain respects, most notably dilator 200 gives a clear indication that the segments, 10, 12, and 14 are in fluid communication with one another. It can also be visually appreciated form the deflated cervical dilator 200′ of FIG. 2 that the respective segments which collectively provide a total length of the dilator may not be distinguishable from one another when the dilator is fully deflated.

In some exemplary embodiments, the cervical dilator (e.g., 100 or 200) is 8-10 cm in length, and in its deflated state it is 7-9 mm in diameter. In some embodiments, the uterine, cervical, and vaginal segments are fluidically connected and are integrated together as a single balloon, though each segment may or may not expand or contract at a different rate than one or both other types of segments, depending on the embodiment. In some exemplary embodiments, the cervical segment 14 varies from 3 to 6 cm in length. All the dimensions provided herein are exemplary for the anatomy of most common adult persons who have a cervix, but other dimensions may be used for some embodiments depending on the intended patient. For example, some humans fall outside the statistical norms and require different sizing than that which is explicitly given in this disclosure. Some embodiments may be used in veterinarian, e.g. equestrian, applications, in which case the cervical dilator may be sized according to the animal on which it is intended to be used, e.g., exotic animal, horse, dog, cat, etc. Similarly, the diameter of the collapsed device for insertion may vary based on the intended final diameter of the middle cervical segment and/or the amount of hydrogel contained.

The entirety of an interior of the cervical dilator 100 or 200, e.g., the collective interiors of the uterine, cervical, and vaginal segments, may be considered a fluid chamber, e.g., a single fluid chamber. As highlighted in the cross-sectional view 14′ (Section B-B) at the top of FIG. 2, the inside of the fluid chamber may contain an expandable material 22 configured to increase in cross-sectional size (in particular, largest cross-sectional dimension, e.g., diameter) at a rate of, e.g., 1-3 mm/hr, or 1-2 mm/hr, when exposed to fluid admitted to inside the chamber, e.g., via valve 16. In this instance the expandable material has a stellate cross-sectional shape which makes contact with the walls 141 of segment 14 at multiple points while leaving gaps 23 through which fluid in the chamber may move. In exemplary embodiments, the expansion of the expandable material 22 is or at least includes expansion in the radial direction, and the increase cross-sectional size is typically an increase in largest cross-sectional dimension, e.g., diameter, particularly in the cervical segment 14. In exemplary embodiments, the wall or walls 141 of the fluid chamber are impermeable to liquids such as water and bodily fluids, so that expansion of the expandable material is controlled only by fluid being added to the cervical dilator balloon by a clinician through an inlet such as the inflation valve 16. That is, external fluid (i.e., fluid outside the dilator) in and around the patient’s cervix does not influence the rate of expansion. Nor is there fluid permeating out of the fluid chamber which could decrease the rate of expansion. The expandable material 22 may be one or more of a hydrogel, a super absorbent polymer, a sodium polyacrylate, and AQUACRYL. AQUACRYL is a copolymer, prepared by a partial alkaline hydrolysis of polyacrylonitrile (PAN) in the presence of sodium thiocyanate (NaSCN). The resulting hydrolysis product is a multi-block acrylic copolymer, containing alternating hydrophilic and hydrophobic blocks. Hydrophilic blocks contain acrylic acid, acrylamidine, and acrylamide. The hydrophobic blocks are formed by the remaining sequences of unreacted acrylonitrile units. The composition of the hydrolysis product varies with the type of AQUACRYL material and depends on reaction conditions and the conversion of the hydrolytic reaction. The expandable material 22 may be in each of the uterine, cervical, and vaginal segments, or it could be only in the cervical segment, or the cervical segment and vaginal segment.

As illustrated in FIGS. 1 and 2, the uterine segment 10 and the vaginal segment 12 may both be larger in diameter than the middle cervical segment 14, at least after partial and/or total inflation. Upon initial inflation of the cervical dilator, the uterine segment 10 may have a smaller volume than the cervical segment 14. The expandable material 22 runs at least the full length of the cervical segment in some embodiments and may even extend into the segments 10 and 12 at either end. In some embodiments, the spaces between the expandable material 22 may be partially or entirely reinforced with ribs to maintain an equal diameter throughout the cervical segment, providing more force to portions more resistant to dilation.

The channel 20, sometimes referred to as a tube, extends the full length of the cervical dilator in exemplary embodiments. In addition to being configured to receive a stylet, the channel 20 also functions to deliver fluid to the uterine segment 10 before the other segments. The fluid chamber, e.g., the combination of the uterine, cervical, and vaginal segments, may be elastic and not under tension when inflated with the fluid, though different segments may have different elasticities from one another. The expandable material 22 may have one or more of a round cross-sectional shape, circular cross-sectional shape, a polygonal cross-sectional shape, a stellate cross-sectional shape, and/or any other appropriate shape.

Ideally the cervical dilator is removable from the patient’s cervix without deflation once the subject has entered the clinic. This may be accomplished simply by pulling on the removal loop 18 (see FIG. 1). FIG. 3 shows an exemplary embodiment in which the inflation valve and removal loop are combined to form a combined device 30. The inflation valve 8 is sealable by a silicone cylinder 32 which is part of one arm 34 of the removal loop. When the silicone cylinder 32 is connected, the combined device 30 serves the function of the removal loop. Thus, when the cervical dilator balloon is to be removed, the clinician simply pulls the combined device 30, but when the cervical dilator balloon is to be inflated, the silicone cylinder 32 is removed to permit access to the inflation valve 8.

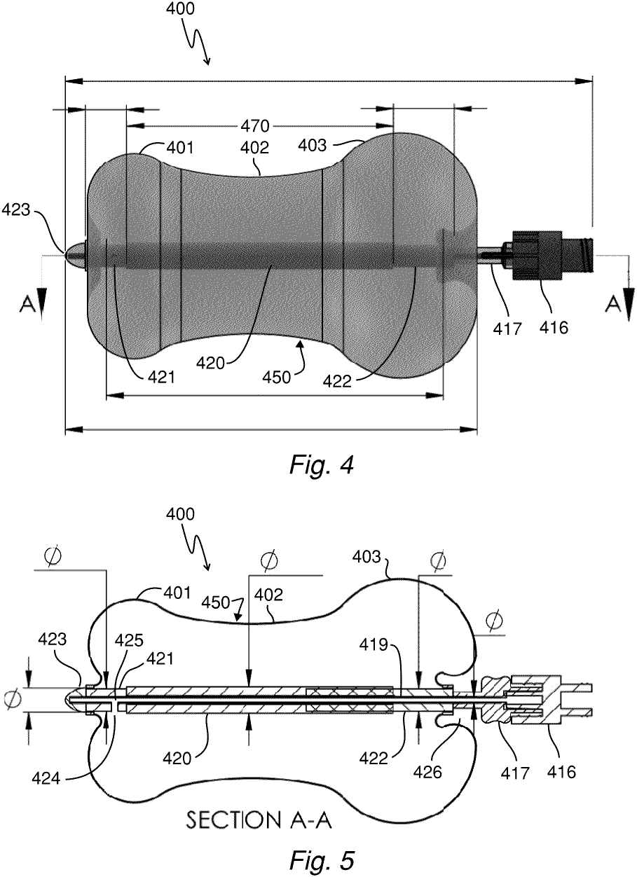

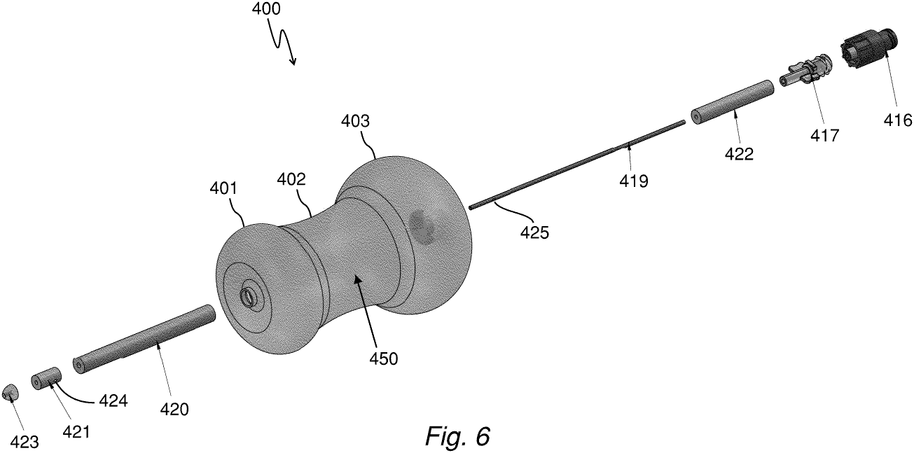

FIG. 4 shows another exemplary dilator 400 from a side view. FIGS. 5 and 6 show the dilator 400 in a cross-sectional view and exploded view, respectively. In all three figures the shape of the fluid reservoir 450 is depicted in an inflated state. However, an expandable material 420, such as but not limited to super absorbent polymer, is depicted in a non-inflated state. The expandable material 420 has a rounded, in particular circular, cross-sectional shape. Other shapes may be employed as an alternative, as discussed above. The balloon 450 includes at least three discernable parts, namely a uterine segment 401, a cervical segment 402, and a vaginal segment 403. The length 470 of expandable material 420 is larger than the length of the cervical segment 402, extending part way (but not fully) into and toward the outer ends of the cervical segment 402 and vaginal segment 403. The fluid chamber/reservoir 450, in particular its walls, encloses the expandable material 420 in a radial direction. The fluid chamber/reservoir may fully enclose the expandable material on all sides.

At either longitudinal end of the expandable material 420 is a tube 421 or 422, and extending through a center through-hole of elements 420, 421, and 422 is a smaller sized tube 419. The tubes 421 and 422 may be polyurethane, for instance. The tube 419 may be stainless steel, for example. The tube 419 terminates at one end in an endpiece 423, which may be a cone of rigid material for example. The tube 419 terminates at the opposite end in a valve for inflation. The valve comprises a first part 416 and a second part 417. The balloon 450 may comprise a depression 426 in the end of the vaginal segment 403 into which part or all of the valve may nest, especially when the valve is not in use.

To inflate the balloon 450 and expose the material 420 to water so that it begins to expand, water is supplied through the valve parts 416/417 to an interior lumen of tube 419. Tube 419 has one or more holes 425 connecting the interior channel/lumen of the tube 419 to an exterior of the tube 419. The placement of the one or more holes 425 controls the initial longitudinal position, or positions, at which the water leaves the tube 419 and enters other spaces within the balloon 450. FIG. 5 shows a hole 425 which aligns with a hole 424 in the tube 421. The holes 425 and 424 permit admission of water directly from the valve to the uterine segment 401. For embodiments in which hole 425 is the only longitudinal side hole in tube 419, the result is admission of water to the uterine segment 401 prior to any admission of water to the cervical segment 402 or vaginal segment 403. This configuration has the advantage of inflating the uterine segment 401 quickly and thus rapidly anchoring the dilator 400. The inflating step causes uterine segment 401 to inflate first with the uterus at a proximal end of the cervix and subsequently to inflate the vaginal segment 403 in the vagina at the distal end of the cervix; the inflated uterine and vaginal segments keep the cervical segment 402 of the device 400 in place inside the cervix.

The dilator 400 differs from the dilators 100 and 200 discussed above in that it does not require a dedicated stylet. The assembly of elements along the center longitudinal axis within the balloon 450 may provide adequate rigidity and stiffness to facilitate placement of the dilator 400, with the valve part 416, or a handpiece connected to valve part 416 (not depicted), serving as the point of contact and manipulation by the clinician.

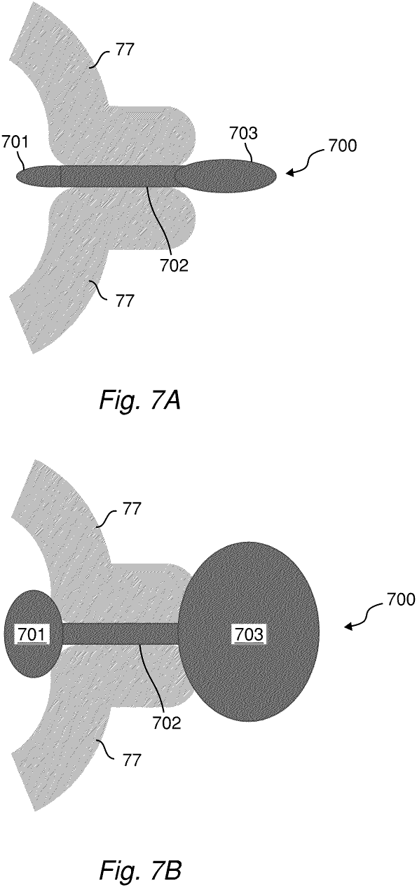

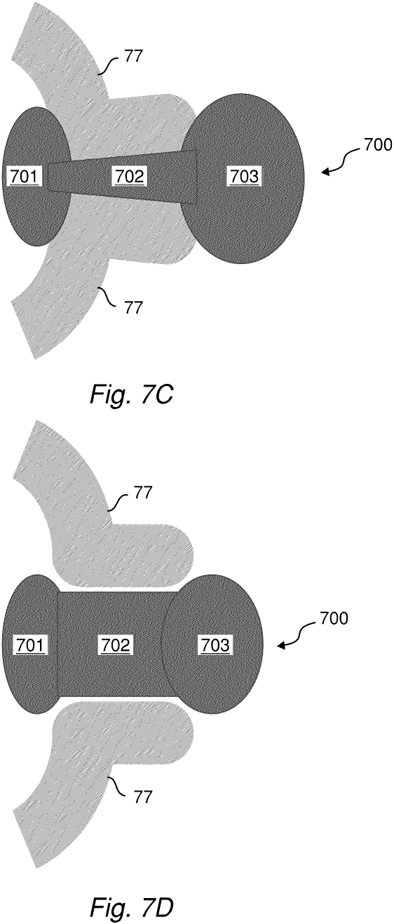

FIGS. 7A, 7B, 7C, and 7D illustrate in stages an exemplary method of dilating a cervix. For simplicity of illustration, the illustrations are limited to depicting the cervical walls 77 and an exemplary cervical dilator 700. When dilating the cervix of a patient is desired, an exemplary procedure employed includes inserting a cervical dilator 700 into a cervix of a subject, as depicted by FIG. 7A. The dilator 700 may take the form of any embodiment within the scope of this disclosure, for instance the devices portrayed in FIGS. 1, 2, or 6, though in their deflated configurations, of which only FIG. 2 shows.) The cervical dilator 700 includes a fluid chamber and an expandable material exposable to fluid in the fluid chamber. The expandable material is configured to increase in largest cross-sectional dimension at a rate of 1, 1-2 or 1-3 mm/hr when exposed to the fluid in the fluid chamber. In FIG. 7B, after insertion, the clinician inflates the cervical dilator 700 with a fixed volume of the fluid, such as 60 ml or more. The fixed volume may vary from 30 to 60 mL, as a means to control or limit final dilation. Then, over several hours (up to 24 or 36 hours) the expandable material in the cervical segment 702 reaches a diameter of at least 3 cm. During the waiting period, the expandable material absorbs or otherwise reacts to the water such that the expandable material slowly expands. FIGS. 7C and 7D depict the changing overall profile of the cervical dilator. Note that in these figures, vaginal segment 703 is reducing in size while the cervical segment 702 is increasing in size. The uterine segment 701 may remain constant or even decrease in size as the cervical segment expands. This is but one example, and the particulars of the overall device profile may vary among embodiments. In embodiments in which the uterine, cervical, and vaginal segments are fluidically connected, in some cases collectively forming a single balloon (e.g., see FIGS. 4-6), all three segments may change in cross-sectional size/dimension and/or internal volume during the waiting period, though each segment may change at a different rate. The waiting period may be done as an in-patient or out-patient, e.g., overnight as discussed above.

In some exemplary embodiments, inflating is performed such that it causes the uterine segment to inflate first with the uterus at a proximal end of the cervix, and, subsequently to inflate the vaginal segment, wherein the inflated uterine and vaginal segments keep the cervical segment of the device in place inside the cervix. In some exemplary embodiments, inflating is not performed with saline or bodily fluids; rather, it is performed with only water, or else water with additives that may improve expansion of the hydrogel. The additive may be, for example, non-ionic agents that improve or control expansion of the hydrogel. Water may be reverse osmosis deionized (RODI) water, distilled water, mineral water, sterile water, and/or tap water, for example. Preferably, in some embodiments, during inflation there is a non-zero pressure on the fluid in the fluid chamber from elastic tension of one or more walls of the fluid chamber. Once the expandable material reaches or exceed a predetermined threshold of e.g. at least 3 cm, as depicted by FIG. 7D, the cervical dilator is removed from the cervix of the subject.

Due to its design and the exemplary process of inflation described above, the inner uterine segment 10 will move the cervical dilator balloon to its proper position on inflation of the uterine segment. For instance, expansion may cause the cervical dilator balloon to pull past the internal cervical os. After the uterine segment is inflated the vaginal segment inflates. Resistance to inflation starts to increase as the vaginal segment 14 creates pressure.

Exemplary cervical dilators may be provided in multiple sizes and shapes (so long as it has uterine segment and vaginal segment that are both enlargeable to a size diameter larger than a cervical segment) to accommodate different subjects. For example, one design may have an uterine segment that is 1-2 cm long, a cervical segment that is 3-4 cm long, and a vaginal segment that is 2-4 cm long, for a total length of 6-9 cm. Kits with a plurality of different sized cervical dilator balloons may include small sizes which inflate to 1.5 to 2 cm in diameter, medium sizes which inflate to 2-3 cm in diameter, and large sizes that inflate from 3-4 cm in diameter.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be separated from or combined with the features of any of the other several embodiments without departing from the scope or spirit of the present invention. Any recited method can be carried out in the order of events recited or in any other order which is logically possible.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

While exemplary embodiments of the present invention have been disclosed herein, one skilled in the art will recognize that various changes and modifications may be made without departing from the scope of the invention as defined by the following claims.

What is claimed is:

1. A medical device for cervical dilation, comprising
a fluid chamber; and
an expandable material exposable to fluid in the fluid chamber,
wherein the expandable material is configured to increase in largest cross-sectional dimension at a rate of 1-3 mm/hr when exposed to the fluid in the fluid chamber.

2. The medical device of claim 1, wherein the fluid is water.

3-6. (canceled)

7. The medical device of claim 1, wherein all walls of the fluid chamber are impermeable to liquids.

8. The medical device of claim 1, wherein the expandable material is a hydrogel.

9. The medical device of claim 8, wherein the expandable material is a superabsorbent polymer.

10. The medical device of claim 1, wherein the expandable material is sodium-polyacrylate.

11. The medical device of claim 1, wherein the fluid chamber has a uterine segment, a vaginal segment, and a cervical segment between the uterine and vaginal segments, wherein the expandable material is positioned within at least the cervical segment.

12. The medial device of claim 11, wherein the uterine, cervical, and vaginal segments are fluidically connected.

13. (canceled)

14. The medical device of claim 11, wherein upon inflation of the fluid chamber with the fluid, the uterine segment and vaginal segment have larger cross-sectional sizes than does the cervical segment.

15. (canceled)

16. The medical device of claim 1, further comprising a tube in a longitudinal center of the medical device surrounded by the expandable material.

17. (canceled)

18. The medical device of claim 16, wherein the tube forms a channel configured to receive a stylet.

19. The medical device of claim 16, wherein the tube forms a channel configured to deliver the fluid to the uterine segment before the other segments.

20-21. (canceled)

22. The medical device of claim 1, further comprising a valve for inflating the fluid chamber and keeping fluid in the fluid chamber.

23-24. (canceled)

25. The medical device of claim 1, wherein the expandable material is configured to expand to a diameter of at least 3 cm in the presence of water.

26-28. (canceled)

29. A method of dilating a cervix, comprising
inserting a cervical dilator into a cervix of a subject, the cervical dilator comprising
a fluid chamber, and
an expandable material exposable to fluid in the fluid chamber,
wherein the expandable material is configured to increase in largest cross-sectional dimension at a rate of 1-3 mm/hr when exposed to the fluid in the fluid chamber;
inflating the cervical dilator with a fixed volume of the fluid;
waiting until the expandable material reaches a largest cross-sectional dimension of at least 3 cm; and
removing the cervical dilator from the cervix after conclusion of the waiting step.

30. The method of claim 29, wherein the fluid used in the inflating step is water, not saline or bodily fluids.

31-35. (canceled)

36. The method of claim 29, wherein the fluid chamber comprises a uterine segment, a vaginal segment, and a cervical segment between the uterine and vaginal segments, wherein the expandable material is positioned within at least the cervical segment, wherein during the waiting step the vaginal segment decreases in size and the cervical segment increases in size.

37. The method of claim 36, wherein the inflating step causes the uterine segment to inflate first in a uterus at a proximal end of the cervix and subsequently to inflate the vaginal segment in a vagina at a distal end of the cervix, wherein the inflated uterine and vaginal segments keep the cervical segment of the device in place inside the cervix.

38. The method of claim 29, wherein the fluid used in the inflating step is water with additional non-ionic agents to improve or control expansion of the hydrogel.

39. The method of claim 29, wherein the method is an out-patient procedure.

40-41. (canceled)
